(19) United States
(12) Patent Application Publication
(10) Pub. No.: US 2024/0335250 A1
(43) Pub. Date: Oct. 10, 2024

(54) Systems and Devices for Enhancing Visibility of Internal Connection Mechanisms

(71) Applicant: Mineral City Biotechnologies, LLC, Jacksonville, FL (US)

(72) Inventors: Chandana Kurra, Fairfield, CT (US); Paul Wasserman, Jacksonville, FL (US); Michael C. Freidl, Jacksonville, FL (US); Travis Meyer, Jacksonville, FL (US)

(21) Appl. No.: 18/224,015

(22) Filed: Jul. 19, 2023

Related U.S. Application Data
(60) Provisional application No. 63/458,027, filed on Apr. 7, 2023.

(51) Int. Cl. A61B 90/00; A61B 17/06; A61L 31/02; A61L 31/14; A61L 31/18

(52) U.S. Cl. CPC A61B 90/39; A61B 17/06166; A61L 31/022; A61L 31/028; A61L 31/145; A61L 31/18; A61B 2090/3954; A61B 2090/3966; A61B 2090/3991

(57) ABSTRACT

Systems and devices for facilitating at least partial visibility of at least one portion of at least one internal connection mechanism within the body of a patient are disclosed. In some aspects, a visibility-enhancing system may comprise at least one visibility-enhancing element and at least one internal connection mechanism. In some implementations, the internal connection mechanism may comprise one or more of: at least one suture, an amount of suture tape, at least one mesh insert, at least one staple, or an amount of tubing. In some embodiments wherein the internal connection mechanism may comprise suture tape, the suture tape may at least partially comprise at least one type of silicone. In some aspects, one or more visibility-enhancing elements may be added to an internal connection mechanism to facilitate at least partial visibility of the internal connection mechanism in one or more medical imaging modalities.

### CROSS-REFERENCE TO RELATED APPLICATION

This application is a Nonprovisional of and claims priority to U.S. Provisional Patent Application Ser. No. 63/458,027 (filed Apr. 7, 2023, and titled “SYSTEMS AND DEVICES FOR ENHANCING VISIBILITY OF INTERNAL CONNECTION MECHANISMS”), the entire contents of which are incorporated herein by reference.

### BACKGROUND

It was nearly 120 years ago that the first X-radiation, or “X-ray,” machine was discovered. X-rays use electromagnetic energy beams to generate imaging of internal bones, organs, and tissue. X-ray beams pass through different parts of the body in varying quantities to produce dark gray pictures of different shades. For example, an X-ray can reveal a broken or fractured bone by passing through the broken or fractured area, appearing as a dark spot within or adjacent to lighter areas that are representative of the bone itself. X-Ray technology has been improved upon and developed since its inception, becoming more sophisticated and contributing to the development of similar diagnostic procedures such as computed tomography (“CT”) scans, fluoroscopy, and arteriograms.

With the common usage of X-ray and other diagnostic machines and procedures, including, for example, magnetic resonance imaging (“MRI”), there is an advantage gained in being able to view, monitor, and trace many features and items, both naturally present and foreign, within a human or animal body. The ability to detect various internal aspects of an organism has facilitated substantial progress in medical treatments. One of the most common treatments in medicine, dating back to ancient Egypt, is suturing. Suturing involves the stitching together of body tissues and/or bones to hold different portions of an incision, wound, breakage, tear, or other injury together to promote healing.

Recently, a new type of suturing material, often referred to as “suture tape” has been developed that has led to even further advancements in making injury repairs, especially those within the body. While suture tape is a very powerful tool, it has recently been discovered that the materials that comprise suture tape do not readily appear within different medical imaging modalities, including X-rays, MRIs, and CT scans. This can be very troublesome for doctors, surgeons, and other healthcare professionals who may rely on accurate knowledge of the location, orientation, and position of suture tape to assess the recovery state of a patient's injury as well as to diagnose any complications related thereto or recommend different types of follow-up care.

### SUMMARY OF THE DISCLOSURE

What is needed are systems and devices for enhancing the visibility of internal connection mechanisms configured within a patient's body. By adding at least one visibility-enhancing element to an internal connection mechanism, medical professionals may be able to more easily locate an internal connection mechanism within the body of a patient using one or more various medical imaging modalities. This may allow medical professionals to provide more comprehensive follow-up care to patients after medical procedures, and ultimately, provide better patient care.

The present disclosure provides for systems and devices for enhancing visibility of internal connection mechanisms within patient bodies. According to some embodiments of the present disclosure, a visibility-enhancing system may comprise at least one visibility-enhancing element and at least one internal connection mechanism. In some aspects, the at least one internal connection mechanism may comprise one or more of: at least one suture, an amount of suture tape, at least one mesh insert, at least one staple, or an amount of tubing, as non-limiting examples. In some non-limiting exemplary embodiments, the at least one internal connection mechanism may comprise an amount of suture tape, wherein the amount of suture tape may comprise at least one type of silicone material. In some embodiments, one or more visibility-enhancing elements May be added to the at least one internal connection mechanism to facilitate at least partial visibility of at least one portion of the at least one internal connection mechanism one or more medical imaging modalities.

In some implementations, at least one visibility-enhancing element may be affixed to, integrated with, embedded within, or otherwise added to at least one internal or external portion of at least one internal connection mechanism. In some aspects, the at least one visibility-enhancing element may comprise one or more of: at least one contrasting agent, an amount of at least one elongated fibrous material in a structural weaving configuration, an amount of at least one elongated fibrous material configured as a tracer line, one or more substantially rigid particles, one or more metallic elements, or an amount of at least one hydrogel, as non-limiting examples. In some non-limiting exemplary embodiments, the at least one visibility-enhancing element may be at least partially visible in one or more medical imaging modalities, such as, by way of example and not limitation, an X-radiation (“X-ray”) image, a magnetic resonance imaging (“MRI”) procedure or the results thereof, a computerized tomography (“CT”) scan or image, or a sonography (“ultrasound”) test or the results thereof. In some implementations, the at least one visibility-enhancing element may be added to the at least one internal connection mechanism during one or more stages of the manufacturing process of the at least one internal connection mechanism, or the at least one visibility-enhancing element may be added to one or more existing internal connection mechanisms after manufacturing has been completed.

### BRIEF DESCRIPTION OF THE DRAWINGS

The accompanying drawings that are incorporated in and constitute a part of this specification illustrate several embodiments of the disclosure and, together with the description, serve to explain the principles of the disclosure:

FIG. 1 illustrates an exemplary system comprising at least one internal connection mechanism, according to some embodiments of the present disclosure.

FIG. 2A illustrates an exemplary visibility-enhancing system comprising at least one internal connection mechanism comprising at least one visibility-enhancing element, according to some embodiments of the present disclosure.

FIG. 2B illustrates an exemplary visibility-enhancing system comprising at least one internal connection mechanism comprising at least one visibility-enhancing element, according to some embodiments of the present disclosure.

FIG. 3 illustrates an exemplary visibility-enhancing system comprising at least one internal connection mechanism comprising at least one visibility-enhancing element, according to some embodiments of the present disclosure.

FIG. 4A illustrates an exemplary medical image of a visibility-enhancing system comprising at least one internal connection mechanism comprising at least one visibility-enhancing element, according to some embodiments of the present disclosure.

FIG. 4B illustrates an exemplary medical image of a visibility-enhancing system comprising at least one internal connection mechanism comprising at least one visibility-enhancing element, according to some embodiments of the present disclosure.

FIG. 5 illustrates an exemplary visibility-enhancing system comprising a plurality of exemplary internal connection mechanisms each comprising at least one visibility-enhancing element, according to some embodiments of the present disclosure.

FIG. 6 illustrates an exemplary visibility-enhancing system comprising at least one internal connection mechanism comprising at least one visibility-enhancing element, according to some embodiments of the present disclosure.

FIG. 7 illustrates an exemplary visibility-enhancing system comprising a plurality of exemplary internal connection mechanisms each comprising at least one visibility-enhancing element, according to some embodiments of the present disclosure.

FIG. 8 illustrates an exemplary data flow process for training an artificially intelligent computing device to detect at least one internal connection mechanism within a body in medical imaging, according to some embodiments of the present disclosure.

### DETAILED DESCRIPTION

The present disclosure provides for systems and devices for facilitating at least partial visibility of at least one portion of at least one internal connection mechanism within the body of a patient, such as a human or animal. According to some embodiments of the present disclosure, a visibility-enhancing system may comprise at least one internal connection mechanism that comprises at least one visibility-enhancing element. In some aspects, the at least one internal connection mechanism may comprise one or more of: at least one suture, an amount of suture tape, at least one mesh insert, at least one staple, or an amount of tubing, as non-limiting examples. In some non-limiting exemplary embodiments wherein the at least one internal connection mechanism may comprise an amount of suture tape, the amount of suture tape may at least partially comprise at least one type of silicone. In some implementations, one or more visibility-enhancing elements may be added to at least one internal connection mechanism to increase visibility of the internal connection mechanism within the body of a patient using one or more medical imaging modalities.

In some aspects, at least one visibility-enhancing element may be affixed to, integrated with, embedded within, or otherwise added to at least one internal or external portion of at least one internal connection mechanism. In some embodiments, the at least one visibility-enhancing element may comprise one or more of: at least one contrasting agent, an amount of at least one elongated fibrous material in a structural weaving configuration, an amount of at least one elongated fibrous material configured as a tracer line, one or more substantially rigid particles, one or more metallic material elements, or an amount of at least one hydrogel, as non-limiting examples. In some aspects, the at least one visibility-enhancing element may be at least partially visible in one or more medical imaging modalities, such as, by way of example and not limitation, an X-radiation (“X-ray”) image, a magnetic resonance imaging (“MRI”) procedure or the results thereof, a computerized tomography (“CT”) scan or image, or a sonography (“ultrasound”) test or the results thereof. In some embodiments, the at least one visibility-enhancing element may be added to the at least one internal connection mechanism during one or more stages of the manufacturing process of the at least one internal connection mechanism, or the at least one visibility-enhancing element may be added to at least one existing internal connection mechanism after the manufacturing process has been completed.

In the following sections, detailed descriptions of examples and methods of the disclosure will be given. The descriptions of both preferred and alternative examples, though thorough, are exemplary only, and it is understood to those skilled in the art that variations, modifications, and alterations may be apparent. It is therefore to be understood that the examples do not limit the broadness of the aspects of the underlying disclosure as defined by the claims.

### Glossary

Internal connection mechanism: as used herein refers to at least one medical material, structure, apparatus, tool, component, or device used internally. In some aspects, an internal connection mechanism may be configured to at least temporarily connect two or more sections or portions of one or more tissues, muscles, bones, or other internal structures or compositions within the body of a patient, such as a human or animal. By way of example and not limitation, an internal connection mechanism may comprise one or more of: at least one suture, an amount of suture tape, at least one mesh structure, at least one staple, at least one bandage or an amount of bandaging, or an amount of tubing. In some non-limiting exemplary embodiments, an internal connection mechanism may be used within an at least partially internal portion of a human or animal body without connecting any structures, sections, or portions. By way of example and not limitation, an internal connection mechanism may comprise one or more of: an amount of gauze or similar absorbent material, a sponge, or amount of sponge material.

Visibility-enhancing element: as used herein refers to any element, structure, substance, composition, or material that may be added to at least one internal or external portion of at least one internal connection mechanism, wherein the visibility-enhancing element may be at least partially visible in at least one medical imaging modality, thereby making the at least one internal connection mechanism easier to locate when the at least one internal connection mechanism is within the body of a patient, such as a human or animal. By way of example and not limitation, a visibility-enhancing element may comprise a form that comprises one or more of: at least one contrasting agent, an amount of at least one fibrous material in a structural weaving configuration, an amount of at least one fibrous material configured as a tracer line, one or more substantially rigid particles or pieces, one or more metallic elements, or an amount of at least one hydrogel that may be integrated with, embedded within, or affixed or applied to one or more internal or external portions of at least one internal connection mechanism. In some non-limiting exemplary embodiments, one or more visibility-enhancing elements may be used within one or more portions of the body of a patient without being affixed to, integrated with, embedded within, or otherwise added to at least one internal connection mechanism.

Additive: as used herein refers to any element that may be added to one or more internal or external portions of at least one internal connection mechanism for one or more purposes other than it in addition to facilitating at least partial visibility of the internal connection mechanism in one or more medical imaging modalities. By way of example and not limitation, an additive may comprise one or more antibiotics, vitamins (such as, for example and not limitation, vitamin E in the form of one or more tocopherols or one or more tocotrienols), an amount of titanium, an amount of iron oxide, an amount of barium sulfate, an amount of manganese chloride or other manganese salt, an amount of aluminum, or one or more agents for enhancing regeneration of orthopedic tissue(s), or one or more nanoparticles comprising any combination thereof. In some aspects, while an additive may not be exclusively intended to facilitate at least partial visibility in one or more medical imaging modalities of at least one internal connection mechanism to which it may be added, an additive may be at least partially visible in medical imaging.

Medical imaging: as used herein refers to any imaging modality that may be used to view one or more locations within the body of a human or animal. In some aspects, medical imaging may comprise radiological imaging that may be used for testing or diagnostic purposes. By way of example and not limitation, medical imaging may comprise X-radiation (“X-ray”), magnetic resonance imaging (“MRI”), computerized tomography (“CT”), or sonography (“ultrasound”).

Fibrous material: as used herein refers to any substance, material, or member that comprises at least one fiber. In some aspects, a fibrous material may comprise one or more of: an amount of thread, an amount of cord, or an amount of string, as non-limiting examples.

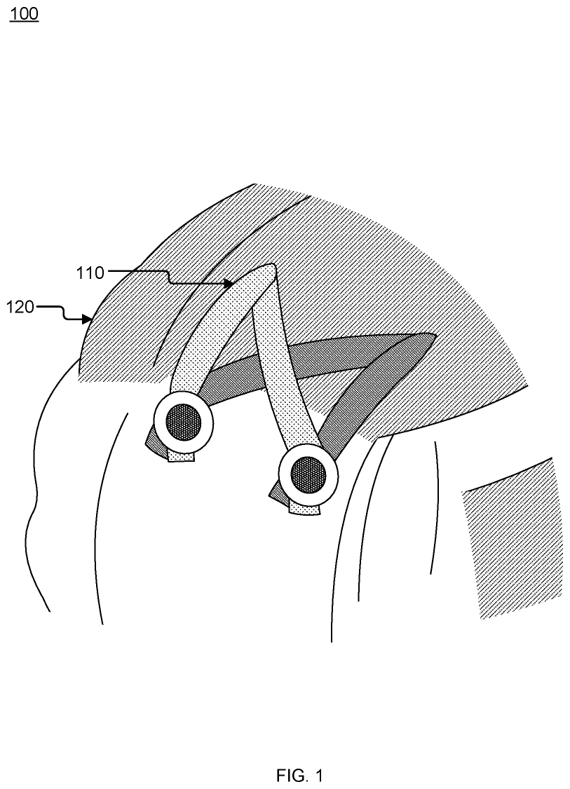

Referring now to FIG. 1, an exemplary system 100 comprising at least one internal connection mechanism 110, according to some embodiments of the present disclosure, is illustrated. In some embodiments, at least one internal connection mechanism 110 may be used to at least temporarily connect two or more internal portions of a patient's body, whether the patient be a human or an animal. In some non-limiting exemplary embodiments, at least one internal connection mechanism 110 may be used to connect two or more portions of one or more bones, muscles, tissues, ligaments, tendons, or similar internal structures or compositions within the body of a patient. By way of example and not limitation, at least one internal connection mechanism 110 may be at least temporarily secured inside a patient to at least one joint, such as a shoulder 120, to tightly hold two or more portions of the joint together to assist with healing after a surgical repair or injury.

In some aspects, the at least one internal connection mechanism 110 may comprise one or more devices or materials that may be substantially invisible or visually imperceptible in one or more medical imaging modalities. For example, the at least one internal connection mechanism 110 may comprise one or more of: at least one suture, an amount of suture tape, at least one mesh insert, at least one staple, an amount of tubing, or any similar device or material that, due its composition or structure, may not be visible via one or more types of medical imaging. For instance, an internal connection mechanism 110 in the form of suture tape may comprise a braided structure that is not visible in X-rays, MRIs, or CT scans. Additionally, an internal connection mechanism 110 may comprise one or more materials that may be substantially translucent or transparent, thereby making them difficult, if not impossible, to see in various types of medical imaging.

In some aspects, the inability to use medical imaging to view the at least one internal connection mechanism 110 within the body of a patient may hinder the ability of a medical care provider to locate the internal connection mechanism 110 during a follow-up visit, potentially causing complications that may be associated with the application, placement, or structural integrity of the internal connection mechanism 110 to go misdiagnosed or undiagnosed completely. For example, any dehiscence of the at least one internal connection mechanism 110 may go undetected until the at least one internal connection mechanism 110 comes apart completely or fails entirely, or an infection associated with the at least one internal connection mechanism 110 may be difficult to treat if the at least one internal connection mechanism 110 cannot be located to be removed. Additionally, an inability to view one or more internal connection mechanisms 110 at or near the site of an upcoming medical procedure, such as a surgery, may cause a medical professional to be required to make one or more adjustments to the procedure as it is being performed, or cancel the procedure altogether, if the internal connection mechanism(s) 110 prove to be a hindrance to the procedure once they are discovered.

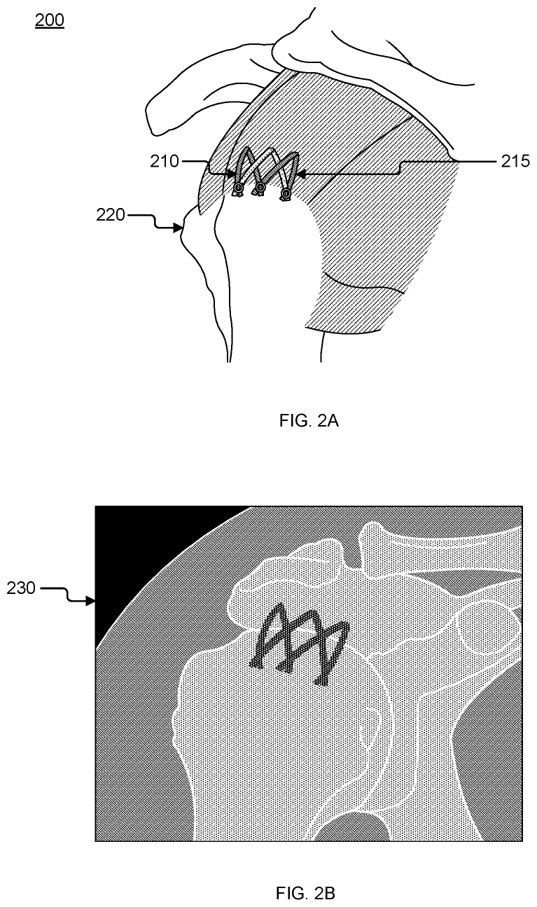

Referring now to FIG. 2A and FIG. 2B, an exemplary visibility-enhancing system 200 comprising at least one internal connection mechanism 210 comprising at least one visibility-enhancing element 215, according to some embodiments of the present disclosure, is illustrated. In some embodiments, at least one internal connection mechanism 210 may be at least temporarily secured within an internal portion of the body of a patient, such as, by way of example and not limitation, by connecting two or more portions of one or more bones, muscles, tissues, ligaments, tendons, or similar structures or compositions within the patient's body. By way of example and not limitation, the at least one internal connection mechanism 210 may be used to connect two or more portions of a joint, such as a shoulder 220, during a medical procedure to facilitate recovery and/or healing. In some aspects of the present disclosure, the at least one internal connection mechanism 210 may comprise one or more devices or materials that may be substantially invisible or visually imperceptible via one or more medical imaging modalities. By way of example and not limitation, the internal connection mechanism 210 may comprise at least one plastic material that comprises one or more of: silicone, ultra-high-molecular-weight polyethylene (“UHMWPE”), nylon, or polypropylene, as non-limiting examples.

In some implementations, the at least one internal connection mechanism 210 may comprise one or more of: at least one suture, an amount of suture tape, at least one mesh insert, at least one staple, or an amount of tubing, as non-limiting examples, the composition or structure of which may cause the at least one internal connection mechanism 210 to not appear in one or more medical imaging modalities, such as, for example and not limitation, an X-ray, an MRI, or a CT scan. In some non-limiting exemplary embodiments, the at least one internal connection mechanism 210 may comprise an amount of suture tape that at least partially comprises at least one type of silicone. In some implementations, if a medical care provider is not be able to locate the at least one internal connection mechanism 210 via medical imaging during a patient's follow-up visit, one or more healing or recovery complications associated with the at least one internal connection mechanism 210 may go misdiagnosed or undiagnosed, or the administration of medical care at or near the location of the at least one internal connection mechanism 210 may be thwarted or delayed.

In some embodiments, at least one visibility-enhancing element 215 may be added to one or more internal or external portions of the at least one internal connection mechanism 210 to facilitate the ability of the internal connection mechanism 210 to be seen in one or more types of medical imaging 230. As a non-limiting example, when the at least one internal connection mechanism 210 comprises at least one visibility-enhancing element 215, the at least one internal connection mechanism 210 may be detected and at least partially visible in medical imaging 230. By way of example and not limitation, a visibility-enhancing element 215 may comprise a form that comprises one or more of: at least one contrasting agent, an amount of at least one elongated fibrous material in a structural weaving configuration, an amount of at least one elongated fibrous material configured as a tracer line, one or more substantially rigid particles, one or more metallic elements, or an amount of at least one hydrogel.

In some non-limiting exemplary embodiments, in aspects wherein the at least one visibility-enhancing element 215 may comprise at least one contrasting agent, the at least one contrasting agent may at least partially comprise an amount of gadolinium, an amount of iodine, or a combination thereof. In some embodiments, at least one visibility-enhancing element 215 in the form of at least one contrasting agent may be added to one or more internal or external portions of an existing internal connection mechanism 210 or may be integrated into or affixed upon one or more internal or external portions of an internal connection mechanism 210 during one or more stages of the manufacturing process of the internal connection mechanism 210.

In some embodiments, at least one visibility-enhancing element 215 may be added as a coating to the at least one internal connection mechanism 210, wherein the at least one visibility-enhancing element 215 may be applied to the at least one internal connection mechanism 210 while in a liquid, resin, or gel form and may solidify to form a coating upon one or more portions of the surface of the at least one internal connection mechanism 210 as it dries. In some implementations, an amount of at least one visibility-enhancing element 215 in a liquid, resin, or gel form may be placed in at least one container, and the at least one container may then be inserted within an internal portion of the at least one internal connection mechanism 210.

In some aspects, an amount of thread, cord, string, or similar elongated fibrous material may be doped with at least one visibility-enhancing element 215 in the form of at least one contrasting agent, and the elongated fibrous material may be used to form structural weaving or braiding that may be integrated within and/or upon at least one portion of at least one internal connection mechanism 210, such as, for example and not limitation, via stitching or sewing, wherein the structural weaving may comprise one or more of a plurality of different patterns or configurations upon and/or within the at least one internal connection mechanism 210. In some embodiments, the at least one visibility-enhancing mechanism 215 may be woven into the at least one internal connection mechanism 210 either during or after the manufacturing of the internal connection mechanism 210.

In some implementations, at least one visibility-enhancing element 215 comprising at least one elongated fibrous material may be integrated with at least one internal connection mechanism 210 so as to physically secure one or more additional visibility-enhancing elements 215 to one or more portions of the at least one internal connection mechanism 210. By way of example and not limitation, a first at least one visibility-enhancing element 215 may comprise at least one elongated fibrous material in a structural weaving configuration that may be configured to at least partially wrap around a second at least one visibility-enhancing element comprising one or more at least partially rigid elongated elements, such as, for example and not limitation, one or more cylindrical or rod-shaped structures, to secure the elongated element(s) to the at least one internal connection mechanism 210, such as, for example and not limitation, an amount of suture tape. In some non-limiting exemplary embodiments, at least one of the one or more elongated elements may at least partially comprise a metallic material to enable the elongated element(s) to be at least partially detected or visible in one or more medical imaging modalities, such as, for example and not limitation, an X-ray.

In some aspects, at least one of the one or more elongated elements may at least partially comprise at least one contrasting agent, such as, for example and not limitation, an amount of gadolinium and/or an amount of iodine, wherein the at least one contrasting agent may be contained within an internal portion of the elongated element(s) or infused with or bonded to one or more portions of the elongated element(s). In some aspects, elongated element(s) that at least partially comprise at least one contrasting agent may comprise one or more at least partially translucent or transparent materials, such as, for example and not limitation, one or more plastics or other polymers.

In some embodiments, at least one visibility-enhancing element 215 comprising at least one additive may be added to the at least one internal connection mechanism 210. By way of example and not limitation, the at least one additive may comprise one or more antibiotics and/or one or more agents for enhancing regeneration of orthopedic tissues. In some aspects, a visibility-enhancing element 215 comprising one or more additives may allow the at least one internal connection mechanism 210 that comprises the at least one visibility-enhancing element to comprise enhanced functionality within the body of a patient, such as by preventing infection or fostering tissue regeneration, as non-limiting examples.

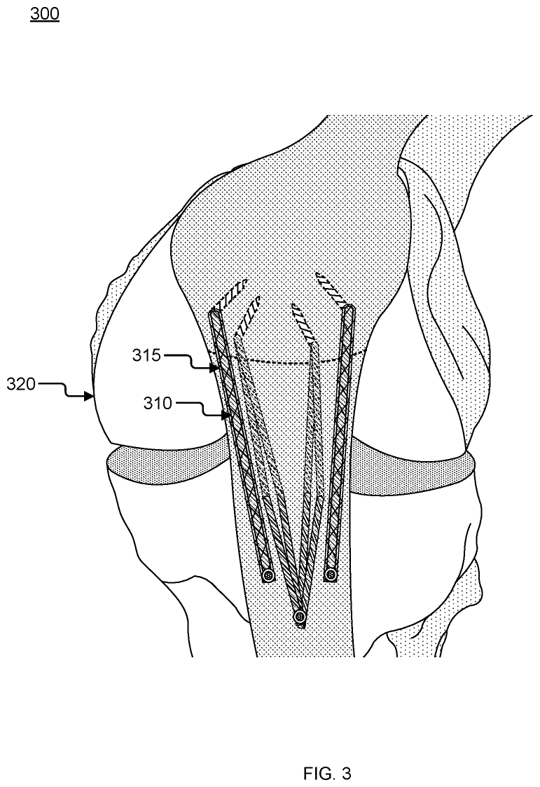

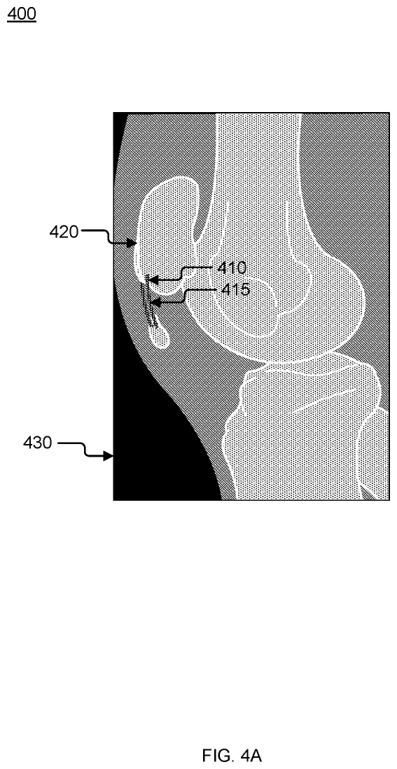

Referring now to FIG. 3, an exemplary visibility-enhancing system 300 comprising at least one internal connection mechanism 310 comprising at least one visibility-enhancing element 315, according to some embodiments of the present disclosure, is illustrated. In some embodiments, at least one internal connection mechanism 310 may be at least temporarily secured at one or more locations within the body of a patient, such as a human or animal. By way of example and not limitation, the at least one internal connection mechanism 310 may be secured during a medical procedure to connect two or more portions of a joint, such as a knee 320. In some aspects of the present disclosure, the at least one internal connection mechanism 310 may comprise one or more devices or materials that may be substantially invisible or visually imperceptible in at least one type of medical imaging.

In some non-limiting exemplary embodiments, an internal connection mechanism 310 may comprise one or more of: at least one suture, an amount of suture tape, at least one mesh insert, a staple, an amount of tubing, or any similar configuration that, due to its structure and/or composition, may make the internal connection mechanism 310 unable to be seen in various types of medical imaging, such as, by way of example and not limitation, an X-ray, an MRI, or a CT scan. By way of example and not limitation, the at least one internal connection mechanism 310 may comprise an amount of suture tape that at least partially comprises at least one type of silicone. In some implementations, various complications may arise if a medical care provider is not be able to locate one or more internal connection mechanisms 310 via medical imaging during a follow-up visit with a patient.

In some embodiments, at least one visibility-enhancing element 315 may be added to one or more internal or external portions of the at least one internal connection mechanism 310 to facilitate the ability of the at least one internal connection mechanism 310 to be seen in one or more medical imaging modalities. In some aspects, by way of example and not limitation, the at least one visibility-enhancing element 315 may comprise one or more of: at least one contrasting agent, an amount of at least one fibrous material in a structural weaving configuration, an amount of at least one fibrous material configured as a tracer line, one or more substantially rigid particles, one or more metallic elements, or an amount of at least one hydrogel. In some non-limiting exemplary embodiments wherein the at least one visibility-enhancing element 315 may comprise at least one contrasting agent, the at least one contrasting agent may at least partially comprise an amount of gadolinium, an amount of iodine, or a combination thereof. In some implementations, the at least one contrasting agent may be added to or integrated with one or more internal or external portions of an internal connection mechanism 310 during or after the manufacturing process of the internal connection mechanism 310.

In some aspects, an amount of thread, cord, string, or similar elongated fibrous material may be doped with at least one visibility-enhancing element 315 in the form of at least one contrasting agent, and the elongated fibrous material may be used to form a mesh overlay that may be integrated with or affixed upon at least one internal connection mechanism 310, wherein the mesh overlay may comprise one or more of a plurality of different patterns or configurations. In some embodiments, the at least one visibility-enhancing mechanism 315 in the form of a mesh overlay may be affixed to one or more portions of an internal connection mechanism 310 during or after the manufacturing process of the internal connection mechanism 310.

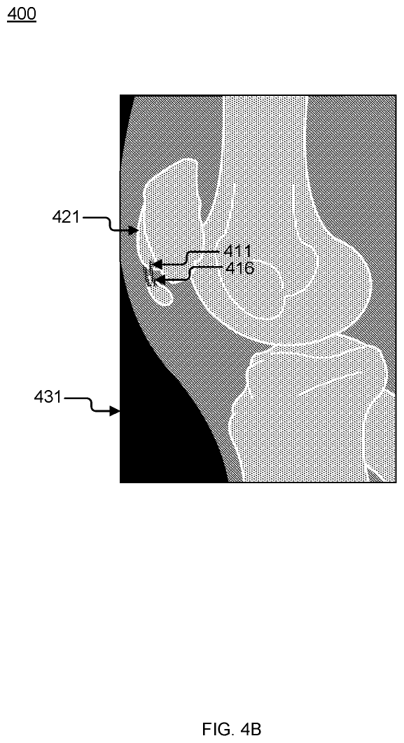

Referring now to FIGS. 4A and 4B, exemplary medical images 430, 431 of an internal connection visibility-enhancing system 400 comprising at least one internal connection mechanism 410, 411 comprising at least one visibility-enhancing element 415, 416, according to some embodiments of the present disclosure, are illustrated. In some embodiments, at least one internal connection mechanism 410, 411 may be at least temporarily secured at one or more locations within the body of a patient, such as a human or animal. By way of example and not limitation, the at least one internal connection mechanism 410, 411 may be secured during a medical procedure to connect two or more portions of a joint, such as a knee 420, 421. In some aspects of the present disclosure, the at least one internal connection mechanism 410, 411 may comprise one or more devices or materials that may be substantially invisible or visually imperceptible in at least one type of medical imaging 430, 431. In some non-limiting exemplary embodiments, the at least one internal connection mechanism 410, 411 may comprise at least one of: at least one suture, an amount of suture tape, at least one mesh insert, at least one staple, or an amount of tubing, or any similar configuration that, due to its composition or structure, may cause the at least one internal connection mechanism 410, 411 to be unable to be seen in one or more types of medical imaging 430, 431, such as, for example and not limitation, an X-ray, an MRI, or a CT scan. By way of example and not limitation, the at least one internal connection mechanism 410, 411 may comprise an amount of suture tape that at least partially comprises at least one type of silicone. In some implementations, various complications may arise if a medical care provider is not be able to locate one or more internal connection mechanisms 410 within a patient via medical imaging 430, 431 during a follow-up visit.

In some embodiments, at least one visibility-enhancing element 415, 416 may be added to the at least one internal connection mechanism 410, 411 to facilitate the ability of the at least one internal connection mechanism 410, 411 to be detected or seen in one or more types of medical imaging 430, 431. In some aspects, the at least one internal connection mechanism 410, 411 may comprise at least one visibility-enhancing element 415, 416 to facilitate the ability of the at least one internal connection mechanism 410, 411 to be detected or seen via one or more modalities of medical imaging 430, 431. In some non-limiting exemplary embodiments, the at least one visibility-enhancing element 415, 416 may comprise one or more of: at least one contrasting agent, an amount of at least one fibrous material in a structural weaving configuration, an amount of at least one fibrous material configured as a tracer line, one or more substantially rigid particles, one or more metallic elements, or an amount of at least one hydrogel. In some implementations wherein the at least one visibility-enhancing element 415, 416 may comprise at least one contrasting agent, the at least one contrasting agent may at least partially comprise an amount of gadolinium, an amount of iodine, or a combination thereof. In some embodiments, a visibility-enhancing element 415, 416 in the form of at least one contrasting agent may be added to an existing internal connection mechanism 410, 411 or may be added to or integrated with at least one internal connection mechanism 410, 411 during the manufacturing process of the at least one internal connection mechanism 410, 411.

In some embodiments, at least one visibility-enhancing element 415, 416 may be added as a coating to the at least one internal connection mechanism 410, 411, wherein the at least one visibility-enhancing element 415, 416 may be applied to the at least one internal connection mechanism 410, 411 while in a liquid, resin, or gel form and may solidify to form a coating upon one or more portions of the surface of the at least one internal connection mechanism 410, 411 as it dries. In some implementations, an amount of at least one visibility-enhancing element 415, 416 in a liquid, resin, or gel form may be placed in at least one container, and the at least one container may then be inserted within an internal portion of the at least one internal connection mechanism 410, 411.

In some aspects, an amount of thread, cord, string, or similar elongated fibrous material may be doped with at least one visibility-enhancing element 415, 416 in the form of at least one contrasting agent, and the elongated fibrous material may be used to form structural weaving or braiding that may be integrated with at least one internal connection mechanism 410, 411, wherein the structural weaving may comprise one or more of a plurality of different patterns or configurations upon and/or within the at least one internal connection mechanism 410, 411. In some embodiments, the at least one visibility-enhancing mechanism 415, 416 may be woven into the at least one internal connection mechanism 410, 411 either during or after the manufacturing of the at least one internal connection mechanism 410, 411.

In some implementations, an internal connection mechanism 410, 411 that comprises at least one visibility-enhancing element 415, 416 may be visible in one or more modalities of medical imaging 430, 431 that may normally not be able to detect or show the at least one internal connection mechanism 410, 411. This may allow one or more medical professionals to monitor or track a patient's healing process, wherein the healing may be facilitated by the at least one internal connection mechanism 410, 411 that comprises at least one visibility enhancing element 415, 416.

As an illustrative example, a patient with an injured knee 420, 421 may seek treatment from an orthopedic surgeon, who may use an internal connection mechanism 410, 411 in the form of suture tape to tightly hold two or more portions of the knee 420, 421 together to facilitate healing of the knee. The suture tape may comprise a visibility-enhancing element 415, 416 in the form of a thread that has been doped with a contrasting agent that comprises a combination of gadolinium and iodine, wherein the thread may have been woven through one or more portions of the suture tape during or after the manufacturing of the suture tape. This may allow the surgeon to be able to locate the suture tape using an X-ray during a follow-up appointment with the patient so that the surgeon may be able to confirm that the patient's knee is healing as intended.

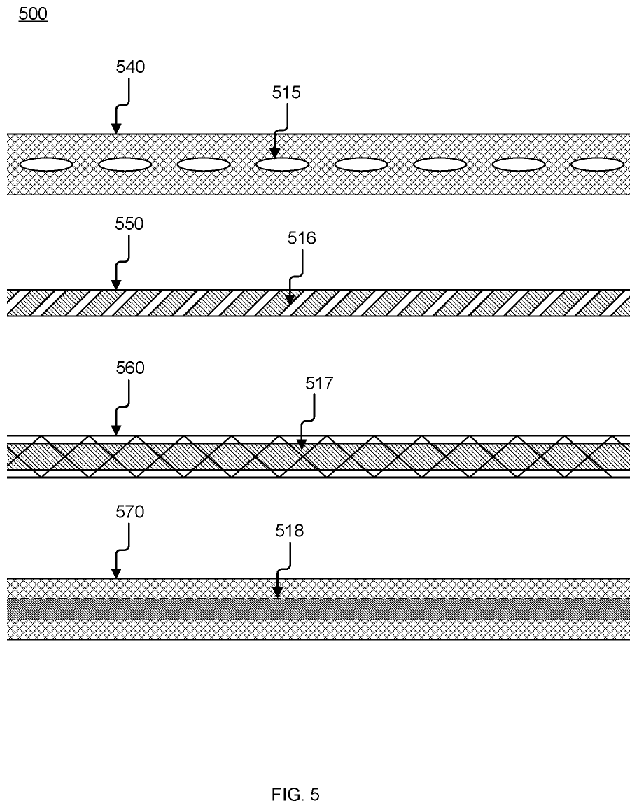

Referring now to FIG. 5, an exemplary visibility-enhancing system 500 comprising a plurality of exemplary internal connection mechanisms 540, 550, 560, 570 each comprising at least one visibility enhancing element 515, 516, 517, 518, according to some embodiments of the present disclosure, is illustrated. In some aspects, an internal connection mechanism 540, 550, 560, 570 may comprise at least one visibility-enhancing element 515, 516, 517, 518 in one or more of a plurality of potential configurations to facilitate the detection and/or visibility of the internal connection mechanisms 540, 550, 560, 570 in one or more medical imaging modalities. In some non-limiting exemplary embodiments, one or more of the exemplary internal connection mechanisms 540, 550, 560, 570 may comprise an amount of suture tape. By way of example and not limitation, one or more of the exemplary internal connection mechanisms 540, 540, 560, 570 may at least partially comprise at least one type of silicone.

In some implementations, an internal connection mechanism 540 may comprise at least one visibility-enhancing element 515 in the form of one or more metallic elements, wherein the metallic element(s) may allow the internal connection mechanism 540 to be detectable and visible via at least one medical imaging modality, such as, for example and not limitation, an X-ray. In some non-limiting exemplary embodiments, the metallic element(s) may be configured as small particles, bands, or staples that may be integrated with or affixed to one or more internal or external portions of an internal connection mechanism 540 during or after the manufacturing of the internal connection mechanism 540. In some aspects, by way of example and not limitation, a visibility-enhancing element 515 that comprises one or more metallic elements may be particularly useful for applications wherein an internal connection mechanism 540 may comprise at least one suture, an amount of suture tape, or a mesh insert.

In some implementations, an internal connection mechanism 550 may comprise at least one visibility-enhancing element 516 in the form of at least one elongated fibrous material in a structural weaving configuration or configured as a tracer line, wherein at least a portion of the elongated fibrous material may be doped with at least one contrasting agent to facilitate its detection or visibility in one or more medical imaging modalities, such as, by way of example and not limitation, an X-ray, MRI, or CT scan. In some non-limiting exemplary embodiments, the at least one elongated fibrous material may comprise an amount of thread, cord, or string that may be integrated with or affixed to one or more internal or external portions of the internal connection mechanism 550 during or after the manufacturing of the internal connection mechanism 550 via stitching, sewing, or weaving, as non-limiting examples. In some aspects, the at least one elongated fibrous material may be configured in one or more of a plurality of potential structural weaving patterns, as a single tracer line, or as a plurality of individual tracer lines or bands, as non-limiting examples.

In some embodiments, an internal connection mechanism 560 may comprise at least one visibility-enhancing element 517 in the form of a mesh overlay, wherein the mesh overlay may comprise one or more fibrous materials arranged in a mesh configuration, wherein at least a portion of the fibrous material(s) may be doped with at least one contrasting agent before the mesh overlay is applied or affixed to one or more portions of the internal connection mechanism 570, wherein the at least one contrasting agent may facilitate the detection and/or visibility of the mesh overlay and the internal connection mechanism 560 with which it may be integrated in one or more medical imaging modalities, such as an X-ray, MRI, or CT scan, as non-limiting examples. In some implementations, by way of example and not limitation, a visibility-enhancing element 517 that comprises a mesh overlay may be applied to one or more portions of an internal connection mechanism 560 during or after a manufacturing process of the internal connection mechanism 560, via stitching, sewing, or adhesion, as non-limiting examples.

In some aspects, an internal connection mechanism 570 may comprise at least one visibility-enhancing element 518 in the form of at least one hydrogel, wherein one or more contrasting agents may be integrated with the at least one hydrogel such that the hydrogel and its associated internal connection mechanism 570 may be at least partially detectable or visible via one or more medical imaging modalities, such as, for example and not limitation, an X-ray, MRI, or CT scan. By way of example and not limitation, a contrasting agent may be integrated with the hydrogel that at least partially comprises iodine, and one iodine moieties may be linked to the hydrogel molecules to bind the contrasting agent to the hydrogel. In some non-limiting exemplary embodiments, a visibility-enhancing element 518 that comprises at least one hydrogel may be added to or embedded or otherwise configured within an internal portion of an internal connection mechanism 570 during or after the manufacturing process of the internal connection mechanism 570. In some implementations, a hydrogel used as a visibility-enhancing element 518 may expand or swell when exposed to one or more fluids within the body of a patient without experiencing dissolution, thereby causing the internal connection mechanism 570 with which it may be integrated to become stronger and form a tighter and more secure connection once it is attached to two or more internal portions, structures, or compositions within the body.

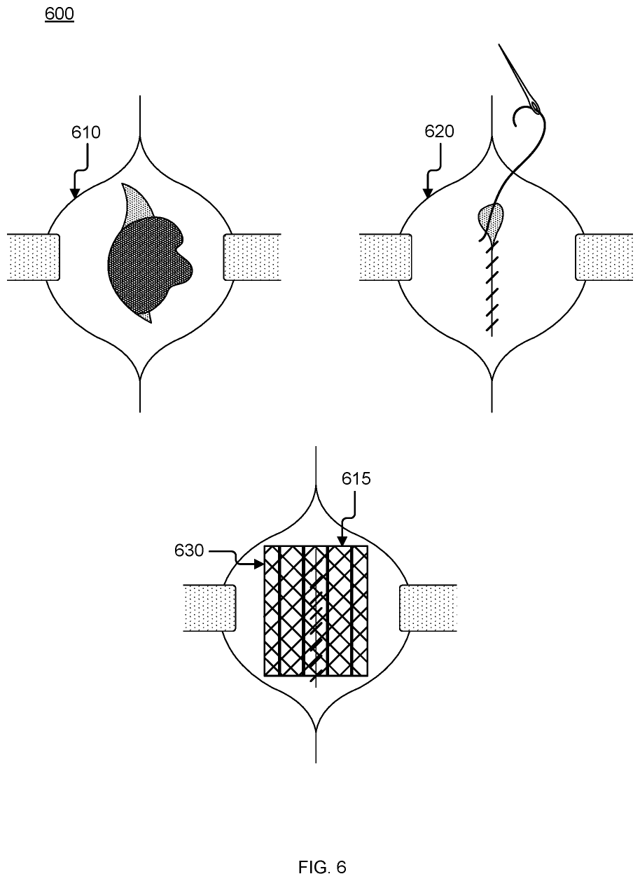

Referring now to FIG. 6, an exemplary visibility-enhancing system 600 comprising at least one internal connection mechanism 630 comprising at least one visibility-enhancing element 615, according to some embodiments of the present disclosure, is illustrated. In some non-limiting exemplary embodiments, at least one internal connection mechanism 630 may be used to facilitate closure and/or healing of at least one incision or wound within an internal portion of the body of a patient. In some implementations, the at least one internal connection mechanism 630 may comprise a mesh insert or other mesh structure or configuration that may be inserted and at least temporarily secured within the body of the patient, wherein the mesh insert may comprise at least one visibility-enhancing element 615 to facilitate detection and/or visibility of the internal connection mechanism 630 in one or more medical imaging modalities, such as, for example and not limitation, an X-ray, MRI, or CT scan. In some aspects, the internal connection mechanism 630 may be configured to remain within the body of the patient indefinitely, as the internal connection mechanism 630 and the at least one visibility-enhancing element 615 integrated therewith may be substantially biocompatible and nondegradable. By way of example and not limitation, the internal connection mechanism 630 may at least partially comprise at least one type of silicone.

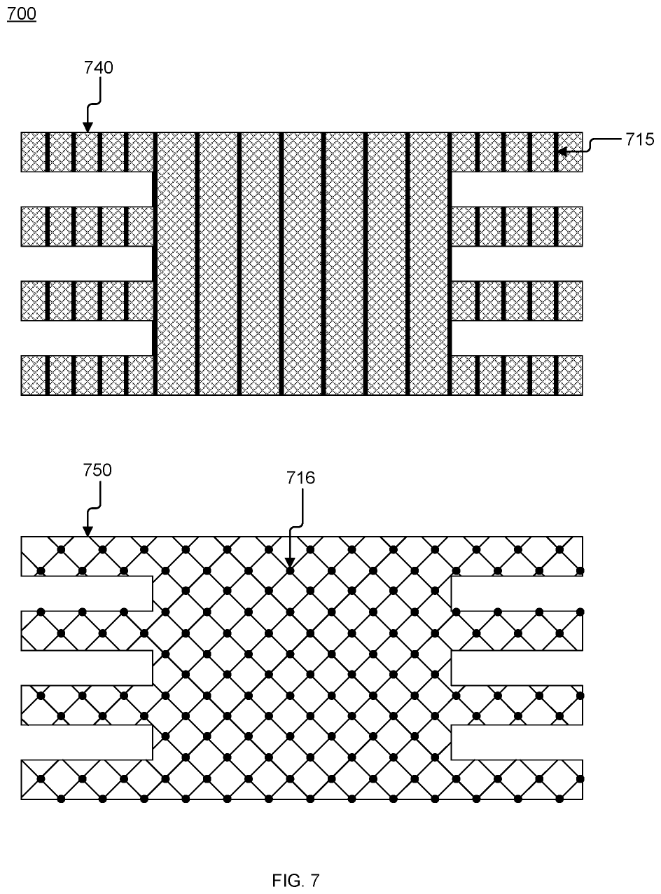

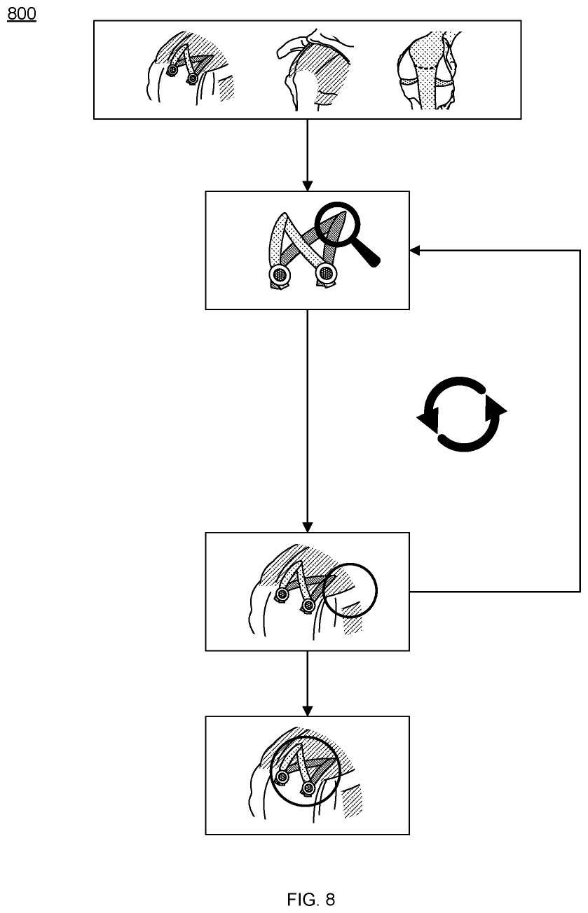

Referring now to FIG. 7, an exemplary visibility-enhancing system 700 comprising a plurality of exemplary internal connection mechanisms 740, 750 each comprising at least one visibility-enhancing element 715, 716, according to some embodiments of the present disclosure, is illustrated. In some aspects, an internal connection mechanism 740, 750 may comprise at least one visibility-enhancing element 715, 716 to facilitate the detection and/or visibility of the internal connection mechanism 740, 750 in one or more medical imaging modalities. In some implementations, the internal connection mechanism 740, 750 may comprise one of a plurality of potential structural configurations. In some non-limiting exemplary embodiments, the internal connection mechanism 740, 750 may comprise one or more fibrous materials and/or one or more plastics, silicones, or other polymers in a mesh configuration.

In some implementations, an internal connection mechanism 740 may comprise at least one visibility-enhancing element 715 in the form of at least one elongated fibrous material in a structural weaving or braided configuration or configured as one or more tracer lines, wherein at least a portion of the elongated fibrous material may be doped with at least one contrasting agent before being integrated with the internal connection mechanism 740 to facilitate the detection or visibility of the elongated fibrous material and the internal connection mechanism 740 associated therewith in one or more medical imaging modalities, such as, by way of example and not limitation, an X-ray, MRI, or CT scan. In some non-limiting exemplary embodiments, the at least one elongated fibrous material may comprise an amount of thread, cord, or string that may be integrated with one or more internal or external portions of the internal connection mechanism 740 via stitching, sewing, or weaving, as non-limiting examples, during or after the manufacturing of the internal connection mechanism 740. In some aspects, the at least one elongated fibrous material may be configured in one or more of a plurality of potential structural weaving patterns or one or more individual tracer lines, as non-limiting examples.

In some implementations, an internal connection mechanism 750 may comprise at least one visibility-enhancing element 716 in the form of one or more substantially rigid particles, wherein at least one of the particles may at least partially comprise one or metallic elements, wherein the metallic element(s) may allow the internal connection mechanism 750 to be detectable and/or visible via at least one medical imaging modality, such as, for example and not limitation, an X-ray. In some non-limiting exemplary embodiments, the one or more substantially rigid particles may be integrated with, affixed to, or embedded within an internal connection mechanism 750 during or after the manufacturing of the internal connection mechanism 750, such as via stitching, sewing, or adhesion, as non-limiting examples. In some aspects, by way of example and not limitation, a visibility-enhancing element 716 that comprises one or more substantially rigid particles may comprise one or more contrasting agents that may be injected or infused into one or more of the particles, or bonded or adhered to an outer surface of one or more of the particles to facilitate detection and/or visibility of the particle(s) and the internal connection mechanism 750 associated therewith in one or more medical imaging modalities.

Referring now to FIG. 8, an exemplary data flow process 800 for training an artificially-intelligent computing device to detect at least one internal connection mechanism within a body in medical imaging, according to some embodiments of the present disclosure, is illustrated. In some aspects, at least one computing device comprising artificial intelligence may be trained to recognize and identify the location of an internal connection mechanism within the body of a human or animal in one or more medical imaging modalities. As a non-limiting example, artificial intelligence may be used to locate a suture attached to shoulder during an ultrasound.

In some embodiments, a computing device comprising artificial intelligence may be trained to identify at least one internal connection mechanism within the body of a patient, such as a human or animal, in one or more medical imaging modalities. By way of example and not limitation, an artificially-intelligent computing device may be provided access to an amount of training data, wherein the training data may comprise a plurality of medical images of a single imaging modality without an internal connection mechanism and a plurality of medical images of the same imaging modality that each comprise at least one internal connection mechanism therein. In some aspects, by analyzing the training data, the artificially-intelligent computing device may develop the ability to distinguish medical images that do comprise at least one internal connection mechanism from those that do not.

In some implementations, a trained artificially intelligent computing device may be tasked with locating at least one internal connection mechanism within the body of a patient in one of a plurality of potential medical imaging modalities. In some embodiments, the artificially intelligent computing device may search an area of the body for the at least one internal connection mechanism using medical imaging. In some aspects, the artificially-intelligent computing device may positively locate the at least one internal connection mechanism that may comprise the target of the search within the body. In some embodiments, the artificially intelligent computing device may use this positive identification to contribute to the training data gathered and stored overtime by the artificially intelligent computing device to enhance the likelihood of finding at least one internal connection mechanism in future searches comprising the same or similar medical imaging modality. In some aspects, overtime, the accuracy and precision with which the artificially-intelligent computing device locates different internal connection mechanisms within patient bodies may increase proportionally to the amount of training data obtained and stored by the artificially-intelligent computing device. In some implementations, the artificially intelligent computing device may determine and present at least one confidence score indicating a likelihood that a particular medical image comprises at least one internal connection mechanism.

In some embodiments, the location of the at least internal connection mechanism identified by an artificially intelligent computing device may be displayed via at least one graphical user interface (GUI) associated with or communicatively coupled to the artificially-intelligent computing device. In some implementations, a user may be able to view the at least one internal connection mechanism within the body of a patient on the GUI. In some aspects, any confidence score(s) associated with the identification of the at least one internal connection mechanism may also be displayed via the GUI.

### CONCLUSION

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination or in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. A visibility-enhancing system, comprising:
at least one suture device; and
where the at least one suture device is comprised of at least one elongated fibrous material;
where the at least one suture device is configured to connect two or more portions of one or more tissues internally within a patient's body;
where the at least one suture device is configured to tightly hold the two or more portions of the one or more tissues together;
where at least one contrasting agent is embedded in the at least one suture device;
where the at least one contrasting agent embedded in the at least one suture device is at least partially visible via at least one medical imaging modality;
where the at least one suture device is comprised of ultra-high-molecular-weight polyethylene (“UHMWPE”), nylon, or polypropylene;
where the least one suture device is within the body of a patient; and
where the at least one suture device is configured by one or more stages of a manufacturing process to include embedded particles of the contrasting agent in the at least one elongated fibrous material within the at least one suture device.

2. The visibility-enhancing system of claim 1, wherein the at least one internal connection mechanism comprises one or more of: at least one suture, an amount of suture tape, at least one mesh structure, at least one staple, or an amount of tubing.

3. The visibility-enhancing system of claim 1, wherein the at least one visibility-enhancing element comprises one or more of: at least one contrasting agent, an amount of at least one fibrous material, one or more substantially rigid particles, and one or more metallic elements.

4. The visibility-enhancing system of claim 1, wherein the at least one contrasting agent at least partially comprises an amount of gadolinium.

5. The visibility-enhancing system of claim 1, wherein the at least one contrasting agent at least partially comprises an amount of iodine.

6. The visibility-enhancing system of claim 3, wherein the at least one visibility-enhancing element comprises the amount of at least one fibrous material doped with the at least one contrasting agent.

7. The visibility-enhancing system of claim 6, wherein the at least one internal connection mechanism comprises an amount of suture tape and the at least one visibility-enhancing element is integrated with at least one portion of the amount of suture tape.

8. The visibility-enhancing system of claim 1, wherein the at least one suture device is integrated internally within a patient's body via stitching.

9. The visibility-enhancing system of claim 1, wherein the at least one suture device visibility enhancing element is integrated with the at least one portion of the suture device visibility-enhancing system to form structural weaving.

10. The visibility-enhancing system of claim 3, wherein the at least one internal connection mechanism comprises an amount of suture tape and the at least one visibility-enhancing element comprises the at least one contrasting agent and the amount of at least one hydrogel.

11. The visibility-enhancing system of claim 10, wherein the at least one contrasting agent is integrated with the amount of at least one hydrogel.

12. The visibility-enhancing system of claim 11, wherein the amount of at least one hydrogel is configured within an internal portion of the amount of suture tape.

13. The visibility-enhancing system of claim 3, wherein the at least one internal connection mechanism comprises a mesh structure.

14. The visibility-enhancing system of claim 13, wherein the at least one visibility-enhancing element comprises the one or more substantially rigid particles.

15. The visibility-enhancing system of claim 14, wherein at least one of the one or more substantially rigid particles comprises at least one of the one or more metallic elements.

16. The visibility-enhancing system of claim 14, wherein the one or more substantially rigid particles are affixed to the mesh structure via stitching or adhesion.

17. The visibility-enhancing system of claim 14, wherein at least one of the one or more substantially rigid particles comprises the at least one contrasting agent.

18. The visibility-enhancing system of claim 1, wherein the at least one medical imaging modality comprises at least one of: at least one X-radiation image, at least one magnetic resonance imaging procedure, or at least one computerized tomography scan.

19. The visibility-enhancing system of claim 1, wherein the at least one internal connection mechanism is configured to at least temporarily connect two or more portions of one or more internal structures within the body of a human or animal.

20. The visibility-enhancing system of claim 19, wherein each of the one or more internal structures comprises one or more of: a bone, a muscle, a tissue, a ligament, or a tendon.

21. A method for generating a visibility-enhancing system internally within a patient's body, the method comprising:
doping an amount of at least one fibrous material with at least one contrasting agent; and
integrating the amount of at least one fibrous material with at least one internal connection mechanism within the patient's body; and
subjecting the patient's body to at least one medical imaging modality, wherein the at least one visibility-enhancing element is at least partially visible via the at least one medical imaging modality.
